(19) United States
(12) Patent Application Publication
(10) Pub. No.: US 2015/0301013 A1
(43) Pub. Date: Oct. 22, 2015

(54) REAL TIME FUEL DISCRIMINATOR

(71) Applicant: James P. Wiebe, Wichita, KS (US)

(72) Inventor: James P. Wiebe, Wichita, KS (US)

(21) Appl. No.: 14/689,012

(22) Filed: Apr. 16, 2015

Related U.S. Application Data
(60) Provisional application No. 61/980,359, filed on Apr. 16, 2014.

(51) Int. Cl. G01N 33/28; G01N 27/22; F02D 41/22

(52) U.S. Cl. CPC G01N 33/2858; F02D 41/22; G01N 33/2847; G01N 27/221

(57) ABSTRACT

A real time fuel discriminator for use with a fuel conveyance system that conveys fuel from a fuel supply to a fuel-powered engine includes a body with an inlet port and an outlet port. Each port is coupled to the fuel conveyance system so that fuel enters the inlet port, passes through the body, and exits the outlet port. A fuel analyzer is associated with the body, and constructed to determine whether the fuel is contaminated with an undesired material. The undesired material may be water or conductive metal fragments.

### CROSS-REFERENCE TO RELATED APPLICATIONS

The present application claims priority to U.S. Provisional Patent Application Ser. No. 61/980,359, filed on Apr. 16, 2014.

### FIELD OF THE INVENTION

The present invention is a device which determines whether a fluid is composed of gasoline, or jet fuel, or water, or contaminated by water.

### BACKGROUND

The detection of a small amount of water within a fluid relies upon measuring the resistance of that fluid, or by measuring the rate of oscillation of a feedback circuit using a capacitive element with the fluid as the dielectric, which in turns changes rate, or frequency, due to a decrease in resistance of the dielectric or the change in dielectric constant.

The detection of a large amount of water relies on identical principles, and in general, will make the dielectric conductive as to disable oscillation and indicate a low resistance value from plate to plate of the dielectric.

The detection of any other fluid is presumed by measuring the rate of oscillation of the feedback circuit, and comparing it known values for various types of fluids. For instance, jet fuel will have a different dielectric value than aviation gasoline, and as a result, their unique presence may be discerned by comparison of oscillation frequencies.

### SUMMARY

In one embodiment of the present invention, a series of capacitors with fluid passing through the dielectric area are utilized, and by electrical analysis of the resistance and/or the capacitance of each capacitor, the invention is able to determine if the fluid is jet fuel, gasoline, water, or a fluid with water contamination. It is also possible to determine if conductive metal fragments are present in the fluid. Furthermore, it is possible to determine whether water contamination is temporarily present or continuously present. Also, consideration is given to the temperature of the fluid. As a result, the dielectric constant, which may vary by temperature, allows a more accurate determination of the type of fluid. Also, variances in the plate area and dielectric thickness of the capacitors allow the capacitors to be created with greater or lesser sensitivity to water contamination and conductive metal fragments, and also to provide preferred dielectric width and size for various fluid types.

### BRIEF DESCRIPTION OF THE DRAWINGS

FIG. 1 is a diagram showing fluid flowing as dielectric between two plates of a capacitor. On the left side of the diagram is the inlet port; on the right side is the outlet port. The capacitor is connected to a circuit through electrical leads from its bottom plate and from its top plate. A electrical symbol for this capacitor is shown, with “F F F” between the plates of the capacitor, and it is given the name “Fluid Capacitor”.

FIG. 2 is a diagram showing an oscillator, with the Fluid Capacitor setting an oscillation frequency based on the dielectric of the fluid. A Schmitt trigger and a feedback resistor are shown, and an electrical symbol is shown for this “Fluid Oscillator”.

FIG. 3 is a diagram showing the addition of a temperature output to the Fluid Oscillator. Such output is determined by the inclusion of a temperature sensor placed in close contact to the flow of fluid within the Fluid Capacitor within the Fluid Oscillator.

FIG. 4 is a diagram showing a microprocessor attached to the Fluid Oscillator. The microprocessor is shown giving an output (or outputs) which may represent “Water”, “Jet Fuel”, “Avgas”, “Ethanol”, “Unknown”, or other fluid types. The microprocessor may have a single output or multiple outputs. The total subcircuit is called “Fluid Oscillator with Analysis and Decision”

FIG. 5 is a diagram showing a microprocessor flow chart which shows an analysis methodology for determining what type of fluid is present. The determination in each decision box is based on oscillation frequency, stability, temperature reference, and defined known values for upper and lower limits thereof.

FIG. 6 is a diagram showing how multiple “Fluid Oscillator with Analysis and Decision” may be run in a serial fashion to determine various types of fluids, where each “Fluid Oscillator with Analysis and Decision” is tuned to its respective type of fluid, with changes in plate area and/or dielectric thickness (width and breadth of fluid channel).

FIG. 7 is a diagram showing how multiple “Fluid Oscillator with Analysis and Decision” may be utilized simultaneously in an aircraft or other vehicle or in other apparatus, so that one is designated “Left”, while another is “Main”, and a third is “Right”. This multiplicity of “Fluid Oscillator with Analysis and Decision” is not limited to three, it may be four or more or many such units. All such units are attached to a “Master” microprocessor, which further analyzes the information from each unit, and the Master microprocessor drives a warning output display.

FIG. 8 is a diagram showing a Master microprocessor flow chart, so that a Left warning triggers a left warning output, a Right warning triggers a right warning output, and a Main triggers a main warning output. If both a Left/Main were active, an additional warning would trigger indicating presence of bad fuel (for instance, water or jet fuel where aviation gasoline was required) from the Left/Main. Equivalently, the Right/Main would trigger a similar alarm.

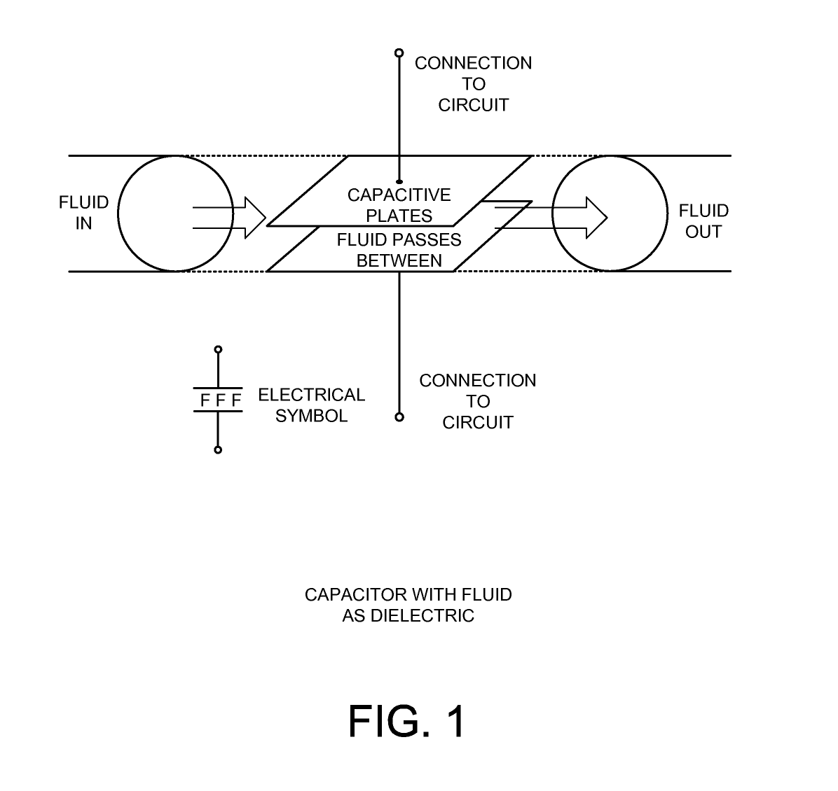

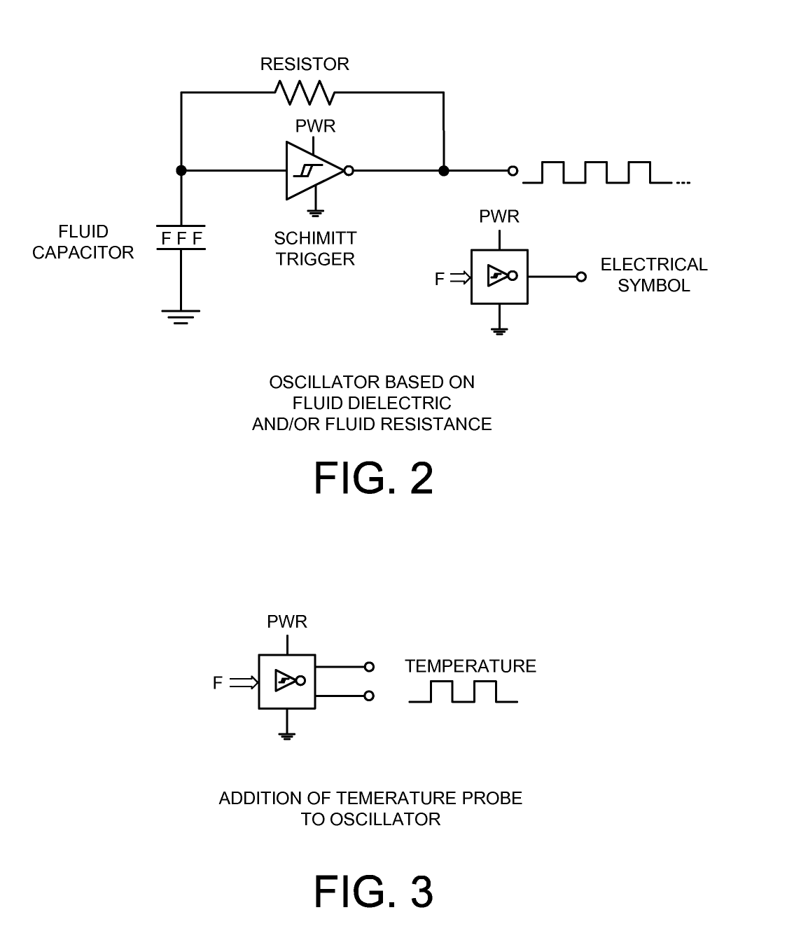

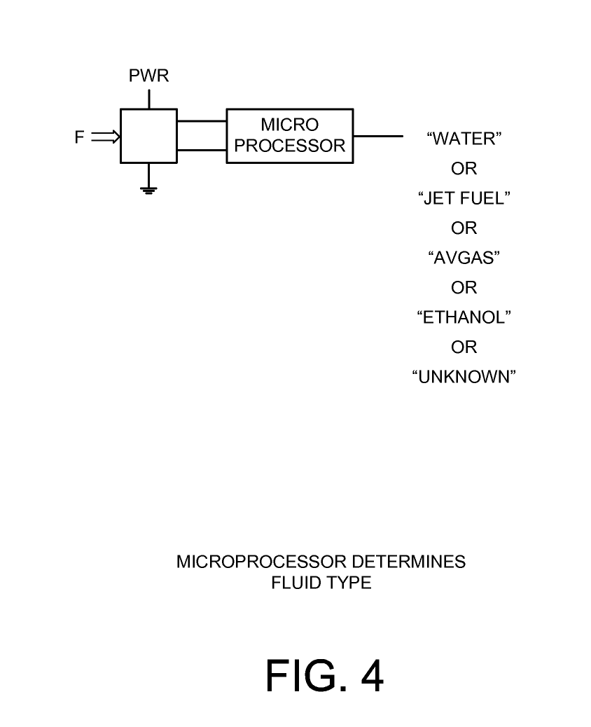

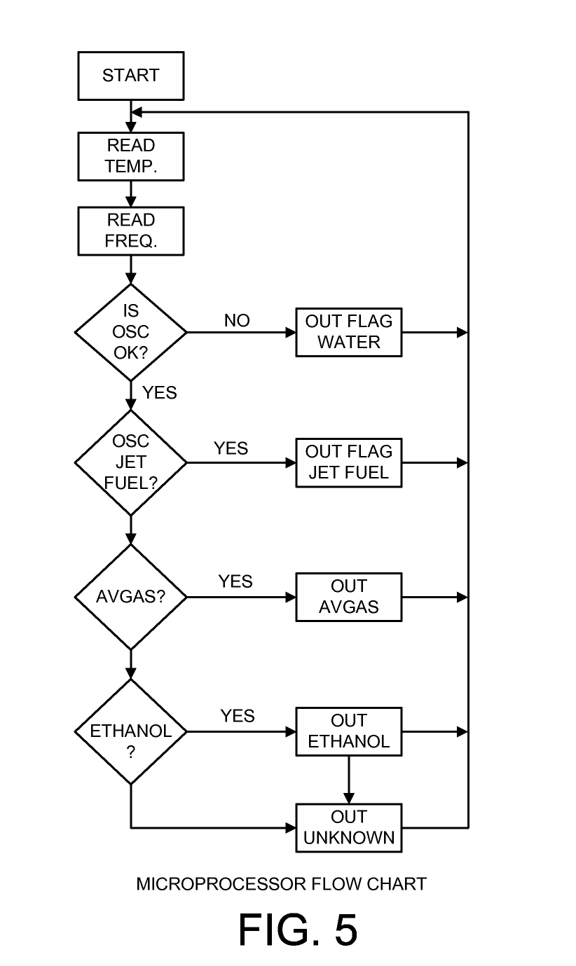

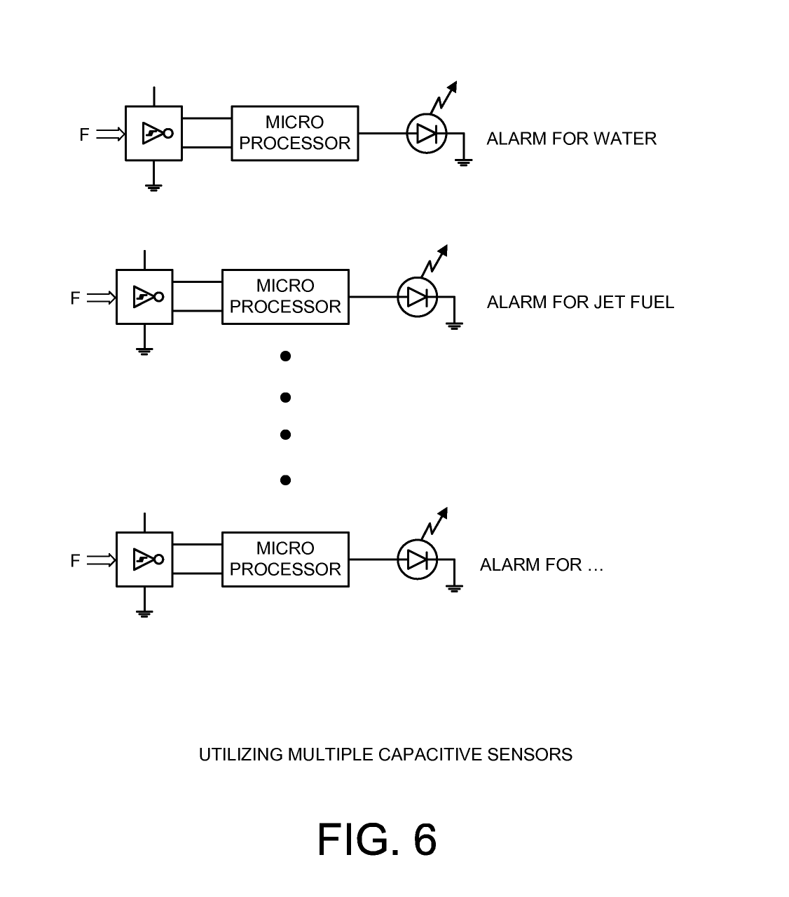

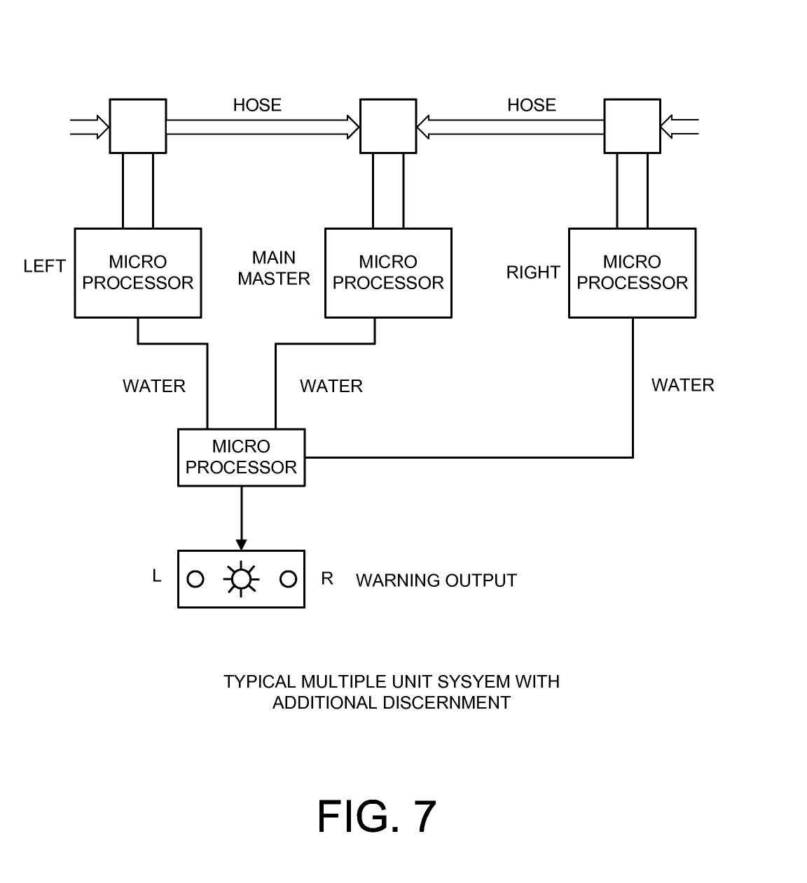

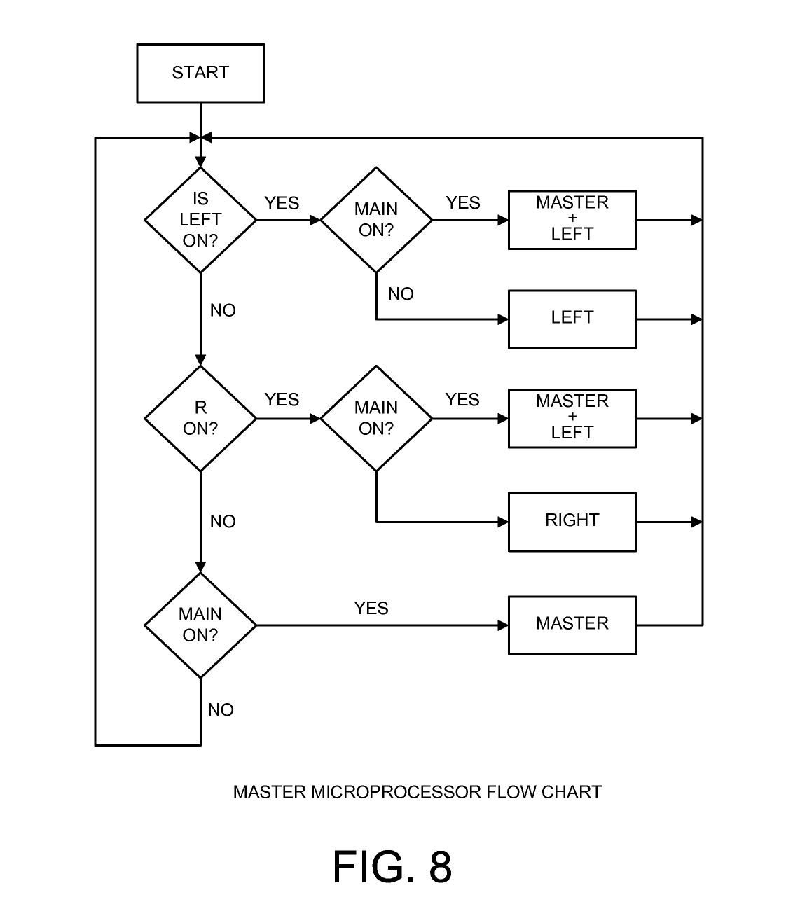

### DETAILED DESCRIPTION

The present invention provides a method determine whether an aircraft (or other vehicle, or other apparatus) has been mis-fueled or has had water contamination. Another way to describe the invention is a real time fuel discriminator for use with a fuel conveyance system that conveys fuel from a fuel supply to a fuel-powered engine includes a body with an inlet port and an outlet port. Each port is coupled to the fuel conveyance system so that fuel enters the inlet port, passes through the body, and exits the outlet port. A fuel analyzer is associated with the body, and constructed to determine whether the fuel is contaminated with an undesired material. The undesired material may be water or conductive metal fragments.

An embodiment of the invention contains the following elements:
10) a capacitor, with fluid flowing between the plates.
12) an inlet to the capacitor, so that fluid may flow to the plates
14) an outlet from the capacitor, so that fluid may flow to the engine (or to a downstream tank, or other apparatus)
16) a connection from the top plate of the capacitor, although the term “top” is not meaningful from an electrical perspective
18) a connection to the bottom plate of the capacitor, although the term “bottom” is not meaningful from an electrical perspective
20) an oscillator circuit, which changes in frequency in response to dielectric changes within the fluid passing through the capacitor
22) a temperature sensor, which provides temperature information to any circuit analyzing the frequency of oscillation. Such sensor is placed close to the flow of fluid, so as to help determine the temperature of that fluid, as dielectric constant changes occur in reference to temperature
24) a microprocessor, which analyzes changes in oscillation frequency correlated with temperature, to determine the type of fluid, or alternatively (in the absence of oscillation) the presence of water
26) a multiplicity of capacitors, oscillators and microprocessors, such that each may be utilized to analyze either for a different type of fluid, or for a different location within a vehicle, such as a left tank, right tank, rear tank, belly tank, main tank, main fuel switch, engine entry position, with each generating an alarm or multiple alarm signals
28) an additional, master microprocessor, which analyzes information coming from individual alarm signals and provides an indication of which tank or fluid path which contains contamination
30) a warning panel or display, which provides a way for the master microprocessor to signal the error
32) a power supply for each element of the circuitry.

10) The capacitor is composed of two metal plates, insulated from each other, within an enclosure which allows fluid to flow from an inlet to an outlet. The capacitor is composed of a substance which is resistant to fluids and corrosion, such as gold plating over a substrate of copper or other conductive surface.

12) The inlet to the fluid capacitor allows attachment to standard fuel line fittings.

14) The outlet of the fluid capacitor allows attachment to standard fuel line fittings.

16) The connection to the top plate of the capacitor allows attachment to an oscillator and microprocessor which is closely located to, or co-located within the same fluid enclosure.

18) The connection to the bottom plate of the capacitor allows attachment to an oscillator and microprocessor which is closely located to, or co-located within the same fluid enclosure.

20) The oscillator circuit is composed of a resistor and Schmitt trigger. Other oscillator circuits may be used, as long as they react appropriately to changes in resistance and/or capacitance in the flow capacitor.

22) The temperature sensor may be of several different commonly available varieties. In all actualities, it merely provides an electrical indication of the fluid temperature.

24) A microprocessor is used in conjunction with each flow capacitor. Such microprocessor is responsible for analysis of the frequency, stability, temperature of the oscillation and determination of fluid type and/or presence of water. The microprocessor must provide an alarm or other signal which is routed to a master microprocessor.

26) The system is composed of a multiplicity of at least two microprocessor analysis units, so that the fuel may be analyzed at the source (such as at the outlet of a fuel tank) and where it enters the engine compartment, or, for example, the fuel switch.

Such multiplicity allows the determination of degraded fuel source, along with an indication of a possibly good tank of fuel.

28) An additional master microprocessor is required to interpret the multiplicity of microprocessors cited in 26), above. The master microprocessor is responsible for providing information to an output warning display. The master microprocessor would be responsible for determining which fluids area allowable (for instance, Jet Fuel) and which are not (For instance, Avgas). In the preponderance of circumstances, water would also not be allowable.

30) The output warning display may be LEDS, or LCDS, or lights, or any other system which utilizes the information provided by the master microprocessor.

32) A power supply is a requirement for all elements of this invention.

Other embodiments could utilization in ground based systems—storage tanks, for instance.

Another embodiment could be in the analysis of contamination in engine oil.

A further embodiment could be the analysis of water contamination in ethanol.

Another embodiment could be the analysis of water contamination in diesel fuel.

The present invention provides a way to determine the type of fluid within a system, and furthermore to determine the type of fluid within several tanks within said system, and furthermore to determine which tanks have water contamination or incorrect fuel, and which fuel paths might possible be uncontaminated. As a result, an operator would be able to manually (or automatically, through microprocessor or system control) change tanks to a correct or uncontaminated source.

What is claimed is:

1. A real time fuel discriminator for use with a fuel conveyance system that conveys fuel from a fuel supply to a fuel-powered engine, comprising:
a body with an inlet port and an outlet port, each port being constructed to be coupled to the fuel conveyance system so that fuel enters the inlet port, passes through the body, and exits the outlet port;
a fuel analyzer associated with the body and constructed to determine whether the fuel is contaminated with an undesired material.

2. The discriminator of claim 1, wherein the undesired material is chosen from the group consisting of water, conductive metal fragments.
